(19) United States
(12) Patent Application Publication
(10) Pub. No.: US 2005/0288374 A1
(43) Pub. Date: Dec. 29, 2005

(54) Chemical stabilization of solubilized retinoids and aqueous compositions comprised thereof

(75) Inventors: Agnes Ferrandis, Mougins (FR); Sandrine Orsoni, Mandelieu (FR); Laurent Fredon, Roquefort Les Pins (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: GALDERMA RESEARCH & DEVELOPMENT, S.N.C., VALBONNE SOPHIA ANTIPOLIS (FR)

(21) Appl. No.: 11/154,655

(22) Filed: Jun. 17, 2005

Related U.S. Application Data
(63) Continuation of application No. PCT/EP03/15041, filed on Dec. 16, 2003.
(60) Provisional application No. 60/437,000, filed on Dec. 31, 2002.

(30) Foreign Application Priority Data
Dec. 17, 2002 (FR) 02/16017

(51) Int. Cl. A61K 31/192

(52) U.S. Cl. 514/568

(57) ABSTRACT

Solubilized retinoids within cosmetic/pharmaceutical compositions, e.g., aqueous gels or oil-in-water emulsions, are chemically stabilized by introducing therein an amount of at least one base effective to salify such retinoids; the resultant compositions are useful in cosmetics and dermatology, e.g., for treating acne vulgaris or psoriasis.

### CROSS-REFERENCE TO PRIORITY/PCT/PROVISIONAL APPLICATIONS

This application claims priority under 35 U.S.C. § 119 of FR 02/16017, filed Dec. 17, 2002, and of provisional application Ser. No. 60/437,000, filed Dec. 31, 2002, and is a continuation of PCT/EP 2003/015041, filed Dec. 16, 2003 and designating the United States (published in the English language on Jul. 1, 2004 as WO 2004/054543 Al), each hereby expressly incorporated by reference and each assigned to the assignee hereof.

### BACKGROUND OF THE INVENTION

1. Technical Field of the Invention:

The present invention relates to the chemical stabilization of a solubilized retinoid within a pharmaceutical composition via addition thereto of a base for salifying the retinoid, and to the aqueous compositions provided thereby.

2. Description of Background and/or Related and/or Prior Art:

It is known that certain retinoids are not readily soluble and lack chemical stability in solubilized form within a pharmaceutical or cosmetic composition (Szuts, “Solubility of retinoids in water”, Arch. Biochem. Biophys., 1991, 287: 297-304). One possible solution to this problem is to incorporate the active ingredient in dispersed form in order to stabilize it. However, a dispersed active ingredient will be released from a topical formulation less easily than a solubilized active ingredient. Thus, to increase the release of the active ingredient, it has proved advantageous to formulate the active ingredient in solubilized form.

Furthermore, a finished product, particularly in the case of pharmaceutical or cosmetic compositions, has to preserve precise physicochemical criteria throughout its life so that its pharmaceutical or cosmetic quality can be guaranteed. Among these criteria, it is necessary for the rheological properties to be preserved. Same define the behavior and texture of the composition on application, as well as the release properties of the principle.

In particular, formulation of the retinoid as a gel or oil-in-water emulsion is advantageous for topical treatments, such as the treatment of acne, especially because it avoids leaving a greasy feel on the skin. Formulation as a water-in-oil emulsion may be preferred for the treatment of psoriasis.

To date, the retinoids according to the invention are not readily soluble and lack stability in oily solvent media as well as in the aqueous solvents compatible with the formulation of a topical composition of the gel or emulsion type.

In WO 85/02767 entitled “Pharmaceutical compositions containing drugs which are unstable or sparingly soluble in water and methods for their preparation”, Janssen Pharmaceutica indicates that “if the molecule has acidic or basic groups, there is the possibility of increasing its solubility in water by the formation of a salt, but this causes a reduction in efficacy or a reduction in chemical stability”. In the light of this prior art, those skilled in the art are not therefore encouraged to salify their active ingredients in order to provide them with chemical stability.

### SUMMARY OF THE INVENTION

A process for the chemical stabilization of solubilized retinoid by the addition of a base for salifying it has now surprisingly been developed, said retinoid becoming soluble and chemically stable within an aqueous composition of the gel or emulsion type. The compositions obtained by the process according to the invention comprise at least one solubilized retinoid which therefore has a good chemical stability, i.e., it does not exhibit degradation of the active ingredient over time at temperatures of from 4° to 40° C.; the composition further has a good physical stability, i.e., it does not exhibit a drop in viscosity over time at temperatures of from 4° and 40° C. and does not exhibit phase separation or an exudate over time at elevated temperature.

Unexpectedly, a process has now been developed which affords excellent chemical stabilization of the solubilized retinoid by salifying it in situ in the aqueous composition by the addition of a base.

The present invention therefore features a process for the chemical stabilization of a solubilized retinoid within an aqueous composition by the addition of a base. This invention also features the aqueous compositions obtained by the process of the invention, comprising, in a physiologically acceptable medium, at least one retinoid and at least one base for salifying the active ingredient, making it possible to solubilize same and provide it with chemical stability.

Advantageously, the aqueous compositions according to the invention comprise an active phase containing the retinoid, a cosolvent and a base for salifying the retinoid, an aqueous phase containing water and optionally another solvent, a gelling agent, additives and an emulsifier in the case of emulsions, and an oily phase in the case of an emulsion, which can also contain coemulsifiers and additives. “Aqueous composition according to the invention” means a composition containing a high percentage of water that is ideally in excess of 50%.

### DETAILED DESCRIPTION OF BEST MODE AND SPECIFIC/PREFERRED EMBODIMENTS OF THE INVENTION

The compositions according to the invention, and more precisely the active phase thereof, contain at least one retinoid, one retinoid precursor or one retinoid derivative.

“Retinoid” means any compound that binds to RAR and/or RXR receptors containing a group capable of forming a salt with a base. “Retinoid precursors” means their immediate biological precursors or substrates, as well as their chemical precursors. “Retinoid derivatives” means both their metabolic derivatives and their chemical derivatives.

The retinoid is preferably a propargyl alcohol derivative and particularly preferably a racemic compound or one of the enantiomers of the formula: 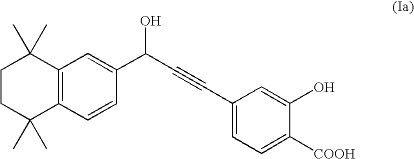
i.e., 2-hydroxy-4-[3-hydroxy-3-(5,6,7,8-tetrahydro-5,5,8,8-tetramethylnaphthalen-2-yl)-1-propynyl]benzoic acid, S-(+)-2-hydroxy-4-[3-hydroxy-3-(5,6,7,8-tetrahydro-5,5,8,8-tetramethylnaphthalen-2-yl)-1-propynyl]benzoic acid or R-(−)-2-hydroxy-4-[3-hydroxy-3-(5,6,7,8-tetrahydro-5,5,8,8-tetramethylnaphthalen-2-yl)-1-propynyl]benzoic acid.

For the purpose of the invention, individual enantiomers of these compounds or mixtures thereof, including racemic mixtures, may be used.

Of course, the amount of retinoid in the composition according to the invention will depend more particularly on the retinoid in question and on the quality of the desired treatment.

The preferred retinoid concentrations are from 0.0001 to 20% by weight, based on the total weight of the composition.

The active phase of the compositions according to the invention also contains a cosolvent of the glycol type or other cosolvents having affinities with the aqueous medium. These hydrophilic solvents, acting as cosolvents, also make it possible to reduce the amount of base and, consequently, to lower the pH compared with that of the glycol-free composition. The pH obtained is therefore closer to that of the skin.

Furthermore, glycols are known to improve the penetration of the active ingredient.

Non-limiting examples of cosolvents which may be mentioned are PEG-6 caprylic/capric glycerides (Softigen 767), nonoxynol-10 (Renex 690), Tween 60, Polysorbate 60, Cremophore RH 60, PEG-35 castor oil, Arlasolve, dimethyl isosorbid, Labrasol, PEG-8 caprylic/capric glycerides, phenoxyethanol, or glycols such as propylene glycol, dipropylene glycol, butylene glycol, polyethylene glycol 400 (PEG-400) and ethoxydiglycol. The preferred cosolvents according to the invention are propylene glycol and dipropylene glycol.

The concentrations of cosolvent in the compositions according to the invention are from 5 to 50% and preferably from 10 to 20%.

The active phase of the compositions according to the invention contains at least one base capable of salifying the retinoid.

Non-limiting examples of bases which may be mentioned are mineral bases such as sodium hydroxide (NaOH) or lithium hydroxide (LiOH), organic bases such as N-methyl-D-glucamine or trometamol, aqueous ammonia (NH₄OH), basic amino acids such as L-lysine, L-arginine, L-ornithine or glycine, or various bases such as D-glucosamine or N-methylglucosamine.

The preferred base of the compositions according to the invention is sodium hydroxide or L-lysine.

The base will preferably be used in a concentration ranging from 0.5 to 10 molar equivalents, based on the retinoid.

The retinoid is solubilized and salified in the presence of the base, i.e.:

a) in the active phase which comprises the cosolvent and the base, simply by magnetic agitation,

b) in the aqueous phase which comprises the solvent, the cosolvent and the base, it also being possible for the aqueous phase to contain additives such as those described herein.

The aqueous phase of the compositions according to the invention contains a solvent such as water, a floral water like cornflower water, or a natural thermal or mineral water selected e.g., from Vittel water, Vichy source waters, Uriage water, Roche Posay water, Bourboule water, Enghien-les-Bains water, Saint Gervais-les-Bains water, Néris-les-Bains water, Allevard-les-Bains water, Digne water, Maizières water, Neyrac-les-Bains water, Lons-le-Saunier water, Bonnes waters, Rochefort water, Saint Christau water, Fumades water, Tercis-les-Bains water, Avène water and Aix-les-Bains water, an alcohol like ethanol, or another hydrophilic solvent.

The preferred solvent is water, which is present in a concentration preferably of more than 50% and particularly preferably of more than 75% in the gel form.

The compositions according to the invention are preferably in the form of aqueous gels.

“Aqueous gel” means a composition which contains, in the aqueous phase, a viscoelastic mass formed from colloidal suspensions (gelling agent).

Non-limiting examples of gelling agents which may be mentioned are acrylic derivatives of the Carbopol type (supplier: Novéon) or Sepigel 305 type (supplier: SEPPIC), cellulosic derivatives of the Natrosol type (supplier: Aqualon) or Methocel type (supplier: Dow Chemical), xanthan gums of the Keltrol type (supplier: KELCO), or mixtures thereof.

The preferred gelling agents are derived from the family of the acrylic derivatives, such as Carbopol 980.

The gelling agent as described above can be used in concentrations preferably ranging from 0.05 to 15% and particularly preferably ranging from 0.1 to 5%.

Another composition according to the invention is an emulsion, which therefore comprises an emulsifying agent within the aqueous phase, and an oily phase.

Non-limiting examples of emulsifying agents which may be mentioned are glyceryl (and) PEG-100 stearate sold under the name Arlacel 165 by ICI or under the name Simulsol 165 by SEPPIC, polyethoxylated fatty acid esters such as Arlatone 983 from ICI, the polyethoxylated (2) stearyl alcohol sold under the name Brij 72, associated with the polyethoxylated (21) stearyl alcohol sold under the name Brij 721 by ICI, sorbitan esters such as the sorbitan oleate sold under the name Arlacel 80 by ICI or sold under the name Crill 4 by Croda, the sorbitan sesquioleate sold under the name Arlacel 83 by ICI or sold under the name Montane 83 by SEPPIC, or sorbitan isostearate, fatty alcohol ethers having a high HLB, i.e., an HLB greater than or equal to 7, such as the ceteareth-20 sold under the name Eumulgin B2 by Cognis or the ceteareth-12 sold under the name Eumulgin B1 by Cognis, or fatty alcohol ethers having a low HLB, i.e., an HLB below 7, such as steareth-2.

The preferred emulsifying agent according to the invention is ceteareth-20.

The compositions according to the invention advantageously comprise up to 15% by weight of an appropriate emulsifying system, preferably from 0.05 to 8% by weight and particularly preferably from 0.1 to 2% by weight, based on the total weight of the composition.

Examples of oily phase components which may be mentioned are oils, especially mineral oils (liquid paraffin), oils of vegetable origin (avocado oil, soya oil), oils of animal origin (lanolin), synthetic oils (perhydrosqualene), silicone oils (cyclomethicone) and fluorinated oils (perfluoropolyethers). Other fatty substances which can be used are fatty alcohols such as cetyl alcohol, fatty acids, waxes and gums, particularly silicone gums.

It is preferable to use liquid paraffins.

The compositions according to the invention can also comprise any additive normally used in the field of cosmetics or pharmaceuticals, such as cyclodextrins, coemulsifiers, sequestering agents, antioxidants, sun filters, preservatives, fillers, electrolytes, humectants, colorants, customary mineral or organic bases or acids, perfumes, essential oils, cosmetic active ingredients, hydrating agents, vitamins, essential fatty acids, sphingolipids, artificial tanning compounds such as DHA, and skin soothing and protecting agents such as allantoin. Of course, those skilled in the art will take care to choose this or these optional complementary compounds and/or their amount in such a way that the advantageous properties of the composition according to the invention are unaffected or substantially unaffected.

These additives can be present in the composition in an amount of 0 to 20% by weight, based on the total weight of the composition.

Examples of cyclodextrins which may be mentioned are β-cyclodextrins or hydroxypropyl-p-cyclodextrins.

Examples of sequestering agents which may be mentioned are ethylenediaminetetraacetic acid (EDTA) and its derivatives or salts, dihydroxyethylglycine, citric acid, tartaric acid or mixtures thereof.

Examples of anti-irritants which may be mentioned are aloe vera, allantoin, oatmeal or tocopherol acetate.

Examples of preservatives which may be mentioned are benzalkonium chloride, phenoxyethanol, benzyl alcohol, diazolidinylurea, parabens or mixtures thereof.

Examples of humectants which may be mentioned are glycerol and sorbitol.

Preferred pH values for the compositions according to the present invention are pH close to the pH of the skin, i.e., between 5 and 7 and preferably between 5,5 and 6.

The present invention therefore features a process for the chemical stabilization of a solubilized retinoid within a pharmaceutical composition by the addition of a base for salifying the retinoid.

In particular, this invention features a process for the chemical stabilization of a solubilized retinoid of the formula: 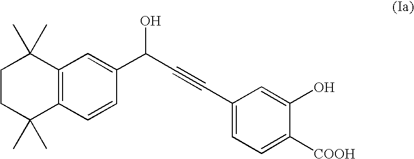
within a pharmaceutical composition by the addition of a base for salifying the retinoid.

The present invention also features pharmaceutical or cosmetic aqueous compositions obtained by the subject process.

In particular, this invention features pharmaceutical or cosmetic aqueous compositions obtained by the subject process for topical application to the skin, integuments or mucosae, in the form of an aqueous gel, wherein it contains the following in a physiologically acceptable medium compatible with topical application to the skin, integuments or mucosae:

a) 0.01 to 5% of a retinoid of the formula:

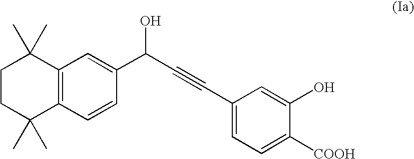

b) 1 to 10 molar equivalents of a mineral base for salifying the retinoid;

c) 0.01 to 5% of an acrylic derivative as a gelling agent;

d) 40 to 80% of water as the main solvent; and

e) 5 to 20% of a glycol as a cosolvent.

A preferred aqueous composition according to the invention comprises:

a) 0.1% of S-(+)-2-hydroxy-4-[3-hydroxy-3-(5,6,7,8-tetrahydro-5,5,8,8-tetramethylnaphthalen-2-yl)-1-propynyl]benzoic acid;

b) 2 molar equivalents of sodium hydroxide for salifying the retinoid;

c) 0.5% of Carbopol 980;

d) 65 to 75% of water; and

e) 15% of propylene glycol.

The present invention also features the compositions as described above as active drugs.

This invention also features the use of the novel compositions as described above in cosmetics and dermatology.

As the compositions according to the invention are especially suited for dermatological use, an important parameter is that of the release and penetration of the active ingredient, which are also improved by way of the formulations provided hereby.

Surprisingly, it has now been found that the preferred formulations described above afford very good results in terms of release and penetration through the skin, which prove to be even better than those afforded by a simple gel containing a high proportion of propenetrating glycol. The compositions obtained according to the invention therefore have a good release/penetration of active ingredient in addition to a good chemical stability of the retinoid.

By virtue of the pronounced activity of retinoids in the fields of cell differentiation and proliferation, the compositions of the invention are particularly suitable in the following therapeutic fields:

1) for treating dermatological afflictions and conditions associated with a keratinization disorder involving differentiation and proliferation, and especially for treating acne vulgaris, comedones, polymorphous acne, acne rosacea, nodulocystic acne, acne conglobata, senile acne, secondary acne such as solar acne, acne medicamentosa or acne keloid, and hidradenitis suppurativa;

2) for treating other types of keratinization disorder and especially ichthyosis, ichthyosiform states, Darier's disease, palmoplantar keratoderma, leukoplakia and leukoplakiform states, and cutaneous or mucous (buccal) lichen;

3) for treating other dermatological afflictions and conditions associated with a keratinization disorder having an inflammatory and/or immunoallergic component, and especially all forms of psoriasis, whether cutaneous, mucous or ungueal, and even psoriatic rheumatism, or atopic dermatitis such as eczema, or respiratory atopy, or gingival hypertrophy; the compounds can also be used for certain inflammatory afflictions or conditions that do not exhibit a keratinization disorder, such as folliculitis;

4) for treating all dermal or epidermal proliferations, whether benign or malignant and whether or not of viral origin, such as common verrucas, plane warts, molluscum contagiosum and epidermodysplasia verruciformis, and oral or florid papillomatosis and proliferations capable of being induced by ultraviolet, especially in the case of actinic keratosis;

5) for repairing or combating skin aging, whether photoinduced or chronological, or for reducing pigmentations, or any pathological conditions associated with chronological or actinic aging;

6) for treating healing disorders or skin ulcers in a preventive or curative capacity, for preventing or repairing striae atrophicae, or for promoting healing;

7) for combating sebaceous gland disorders such as acneform hyperseborrhoea or simple seborrhoea;

8) in the treatment of any skin afflictions or conditions of fungal origin, such as tinea pedis and tinea versicolor;

9) in the treatment of dermatological afflictions and conditions having an immunological component;

10) in the treatment of skin disorders due to exposure to UV radiation; and

11) in the treatment of dermatological afflictions and conditions associated with an inflammation or infection of the tissues surrounding the hair follicle, especially those due to a microbial colonization or infection, particularly impetigo, seborrhoeic dermatitis, folliculitis or sycosis barbae, or those involving any other bacterial or fungal agent.

The compositions according to the invention are particularly suitable for the preventive or curative treatment of acne vulgaris or psoriasis, whether regime or regimen.

The compositions according to the invention are also administered in the field of cosmetics, particularly for treating skin prone to acne, for causing hair regrowth or preventing hair loss, for combating the greasy appearance of the skin or hair, in providing protection from the harmful effects of the sun, in treating physiologically dry skin or for preventing and/or combating photoinduced or chronological aging.

The compositions according to the invention are also useful in body and hair hygiene.

The present invention also features the use of a base for the chemical stabilization, by salification, of a solubilized retinoid in a pharmaceutical composition comprising at least one retinoid, a principal solvent and a cosolvent.

In order to further illustrate the present invention and the advantages thereof, the following specific examples are given, it being understood that same are intended only as illustrative and in nowise limitative. In said examples to follow, all parts and percentages are given by weight, unless otherwise indicated. Results pertaining to physical and chemical stability and to the release and penetration of the active ingredient are also given by way of illustration.

### EXAMPLE 1

### Process for the Chemical Stabilization of the Solubilized Retinoid within the Pharmaceutical Compositions According to the Invention

The retinoid is solubilized and salified in the presence of the base, i.e.:

a) in the active phase comprising the cosolvent and the base, simply by magnetic agitation;

b) in the aqueous phase comprising the solvent, the cosolvent and the base, it also being possible for the aqueous phase to contain additives such as those described herein.

The examples which follow relate to compositions whose active phase and/or aqueous phase are prepared by the process described.

### EXAMPLE 2

### Aqueous Gel

| COMMERCIAL NAME | INCI NAME | % |
|---|---|---|
| WATER | Water | 75.88 |
| METHYL PARABEN | Methyl paraben | 0.15 |
| GLYCEROL | Glycerin | 5.00 |
| ALLANTOIN | Allantoin | 0.20 |
| EDTA 2 Na | Sodium edetate | 0.10 |
| CARBOPOL 980 NF | Carbomer | 0.50 |
| SODIUM HYDROXIDE (10% | Sodium hydroxide | 2.00 |
| SOLUTION) | | |
| PROPYLENE GLYCOL | Propylene glycol | 15.00 |
| S-(+)-2-hydroxy-4-[3-hydroxy-3-(5,6,7,8- | Active ingredient | 0.10 |
| tetrahydro-5,5,8,8- | | |
| tetramethylnaphthalen-2-yl)-1- | | |
| propynyl]benzoic acid | | |
| SODIUM HYDROXIDE (1% SOLUTION) | Sodium hydroxide | 1.07 |

Procedure:

Active phase: This is prepared by the process of Example 1a) by magnetic solubilization of the propylene glycol, the sodium hydroxide and the retinoid.

Aqueous phase: Under the action of heat (80° C.), assure perfect solubilization of the methyl paraben, the glycerol, the allantoin and the EDTA.

Then assure perfect dispersion of the Carbopol in this phase. Neutralize the gel and incorporate the active phase into it.

### EXAMPLE 3

### Aqueous Gel with Cyclodextrins

| COMMERCIAL NAME | INCI NAME | % |
|---|---|---|
| WATER | Water | 40.43 |
| METHYL PARABEN | Methyl paraben | 0.15 |
| GLYCEROL | Glycerin | 5.00 |
| CARBOPOL 980 NF | Carbomer | 0.50 |
| SODIUM HYDROXIDE (10% SOLUTION) | Sodium hydroxide | 2.00 |
| WATER | Water | 45.00 |
| PROPYLENE GLYCOL | Propylene glycol | 5.00 |
| β-CYCLODEXTRINS | Cyclodextrin | 0.75 |
| SODIUM HYDROXIDE (1% SOLUTION) | Sodium hydroxide | 1.07 |
| S-(+)-2-hydroxy-4-[3-hydroxy-3-(5,6,7,8- | Active ingredient | 0.10 |
| tetrahydro-5,5,8,8-tetramethylnaphthalen- | | |
| 2-yl)-1-propynyl]benzoic acid | | |

Procedure:

The active phase, which in this case is also the aqueous phase, is prepared by the process of Example 1b) by solubilization of the retinoid in the presence of the aqueous sodium hydroxide solution, the propylene glycol and the cyclodextrins.

### EXAMPLE 4

### Oil-in-Water Emulsion

| COMMERCIAL NAME | INCI NAME | % |
|---|---|---|
| MARCOL 172 | Mineral oil | 10.00 |
| EUMULGIN B2 | Ceteareth-20 | 0.50 |
| BHT | Butyl hydroxy toluene | 0.05 |
| PROPYL PARABEN | Propyl paraben | 0.15 |
| WATER | Water | 65.78 |
| GLYCEROL | Glycerin | 5.00 |
| ALLANTOIN | Allantoin | 0.20 |
| CARBOPOL 980 NF | Carbomer | 0.15 |
| PEMULEN TR1 | Acrylates/C10-30 alkyl | 0.30 |
| | acrylate crosspolymer | |
| SODIUM HYDROXIDE (10% | Sodium hydroxide | 1.70 |
| SOLUTION) | | |
| PROPYLENE GLYCOL | Propylene glycol | 15.00 |
| S-(+)-2-hydroxy-4-[3-hydroxy-3- | Active ingredient | 0.10 |
| (5,6,7,8-tetrahydro-5,5,8,8- | | |
| tetramethylnaphthalen-2-yl)-1- | | |
| propynyl]benzoic acid | | |
| SODIUM HYDROXIDE (1% | Sodium hydroxide | 1.07 |
| SOLUTION) | | |

Procedure:

Active phase: This is prepared by the process of Example 1a) by magnetic solubilization of propylene glycol, the sodium hydroxide and the retinoid.

Aqueous phase: Weigh the water, the glycerol and the allantoin into the formulating beaker and raise the temperature to 80° C.

Assure perfect solubilization of the Carbopol and the Pemulen, with Rayneri agitation.

Oily phase: Weigh the oily phase comprising the Marcol 172, the Eumulgin B2, the BHT and the propyl paraben and raise the temperature to 80° C.

Carry out the emulsification at 80° C. for 20 min, with Rayneri agitation, and then cool gradually to 50°.

At 50° C., neutralize the gelling agents and add the active phase, with agitation.

### EXAMPLE 5

### Oil-in-Water Emulsion

| COMMERCIAL NAME | INCI NAME | % |
|---|---|---|
| MARCOL 172 | Mineral oil | 10.00 |
| EUMULGIN B2 | Ceteareth-20 | 0.50 |
| BHT | Butyl hydroxy toluene | 0.05 |
| WATER | Water | qs 100 |
| GLYCEROL | Glycerin | 5.00 |
| CARBOPOL 980 NF | Carbomer | 0.2 |
| PHENOXYETHANOL | Phenoxyethanol | 1.00 |
| PEMULEN TR1 | Acrylates/C10-30 alkyl | 0.30 |
| | acrylate crosspolymer | |
| SODIUM HYDROXIDE (10% | Sodium hydroxide | 1.80 |
| SOLUTION) | | |
| DIPROPYLENE GLYCOL | Dipropylene glycol | 15.00 |
| S-(+)-2-hydroxy-4-[3-hydroxy-3- | Active ingredient | 0.10 |
| (5,6,7,8-tetrahydro-5,5,8,8- | | |
| tetramethylnaphthalen-2-yl)-1- | | |
| propynyl]benzoic acid | | |
| SODIUM HYDROXIDE (1% | Sodium hydroxide | 1.07 |
| SOLUTION) | | |

Procedure:

Active phase: This is prepared by the process of Example 1a) by magnetic solubilization of the dipropylene glycol, the sodium hydroxide and the retinoid.

Aqueous phase: Weigh the water, the glycerol and the phenoxyethanol into the formulating beaker and raise the temperature to 80° C.

Assure perfect solubilization of the Carbopol and the Pemulen, with Rayneri agitation.

Oily phase: Weigh the oily phase comprising the Marcol 172, the Eumulgin B2 and the BHT and raise the temperature to 80° C.

Carry out the emulsification at 80° C. for 20 min, with Rayneri agitation, and then cool gradually to 50°.

At 50° C., neutralize the gelling agents and add the active phase, with agitation.

### EXAMPLE 6

### Aqueous Gel

| COMMERCIAL NAME | INCI NAME | % |
|---|---|---|
| WATER | Water | 75.93 |
| METHYL PARABEN | Methyl paraben | 0.15 |
| BHT | Butyl hydroxy toluene | 0.05 |
| GLYCEROL | Glycerin | 5.00 |
| ALLANTOIN | Allantoin | 0.20 |
| CARBOPOL 980 NF | Carbomer | 0.50 |
| SODIUM HYDROXIDE (10% | Sodium hydroxide | 2.00 |
| SOLUTION) | | |
| PROPYLENE GLYCOL | Propylene glycol | 15.00 |
| S-(+)-2-hydroxy-4-[3-hydroxy-3- | Active ingredient | 0.10 |
| (5,6,7,8-tetrahydro-5,5,8,8- | | |
| tetramethylnaphthalen-2-yl)-1- | | |
| propynyl]benzoic acid | | |
| SODIUM HYDROXIDE (1% | Sodium hydroxide | 1.07 |
| SOLUTION) | | |

Procedure:

Active phase: This is prepared by the process of Example 1a) by magnetic solubilization of the propylene glycol, the sodium hydroxide and the retinoid.

Aqueous phase: Under the action of heat (80° C.), assure perfect solubilization of the methyl paraben, the glycerol, the allantoin and the BHT. Then assure perfect dispersion of the Carbopol in this phase. Neutralize the gel and incorporate the active phase into it.

### EXAMPLE 7

### Aqueous Gel

| COMMERCIAL NAME | INCI NAME | % |
|---|---|---|
| WATER | Water | 75.93 |
| METHYL PARABEN | Methyl paraben | 0.15 |
| BHT | Butyl hydroxy | 0.05 |
| GLYCEROL | toluene | 5.00 |
| ALLANTOIN | Glycerin | 0.20 |
| CARBOPOL 980 NF | Allantoin | 0.50 |
| | Carbomer | |
| SODIUM HYDROXIDE (10% | Sodium hydroxide | 2.00 |
| SOLUTION) | | |
| DIPROPYLENE GLYCOL | Dipropylene glycol | 15.00 |
| S-(+)-2-hydroxy-4-[3-hydroxy-3-(5,6,7,8- | Active ingredient | 0.10 |
| tetrahydro-5,5,8,8- | | |
| tetramethylnaphthalen-2-yl)-1- | | |
| propynyl]benzoic acid | | |
| SODIUM HYDROXIDE (1% SOLUTION) | Sodium hydroxide | 1.07 |

Procedure:

Active phase: This is prepared by the process of Example 1a) by magnetic solubilization of the dipropylene glycol, the sodium hydroxide and the retinoid.

Aqueous phase: Under the action of heat (80° C.), assure perfect solubilization of the methyl paraben, the glycerol, the allantoin and the BHT.

Then assure perfect dispersion of the Carbopol in this phase. Neutralize the gel and incorporate the active phase into it.

### EXAMPLE 8

### Aqueous Gel

| COMMERCIAL NAME | INCI NAME | % |
|---|---|---|
| WATER | Water | 75.93 |
| METHYL PARABEN | Methyl paraben | 0.15 |
| BHT | Butyl hydroxy | 0.05 |
| GLYCEROL | toluene | 5.00 |
| ALOE VERA | Glycerin | 0.20 |
| CARBOPOL 980 NF | Aloe vera | 0.50 |
| | Carbomer | |
| SODIUM HYDROXIDE (10% | Sodium hydroxide | 2.00 |
| SOLUTION) | | |
| DIPROPYLENE GLYCOL | Dipropylene glycol | 15.00 |
| 2-Hydroxy-4-[3-hydroxy-3-(5,6,7,8- | Active ingredient | 0.10 |
| tetrahydro-5,5,8,8- | | |
| tetramethylnaphthalen-2-yl)-1- | | |
| propynyl]benzoic acid | | |
| SODIUM HYDROXIDE (1% SOLUTION) | Sodium hydroxide | 1.07 |

Procedure:

Active phase: This is prepared by the process of Example 1a) by magnetic solubilization of the dipropylene glycol, the sodium hydroxide and the retinoid.

Aqueous phase: Under the action of heat (80° C.), assure perfect solubilization of the methyl paraben, the glycerol, the aloe vera, the BHT and the water.

Then assure perfect dispersion of the Carbopol in this phase. Neutralize the gel and incorporate the active phase into it.

### EXAMPLE 9

### Stability

### 9.A. Chemical Stability of 2-hydroxy-4-[3-hydroxy-3-(5,6,7,8-tetrahydro-5,5,8,8-tetramethylnaphthalen-2-yl)-1-propynyl]benzoic acid (hereafter Referred to as Active Ingredient in this Example) in a Simple Glycolic Gel with and without Salification

Five simple compositions were prepared for the purpose of verifying the chemical stability of the active ingredient with or without salification.

Details of the compositions of the formulae:

| Ingredient | Formulation | Formulation | Formulation | Formulation | Formulation |
|---|---|---|---|---|---|
| (%) | no. 1 | no. 2 | no. 3 | no. 4 | no. 5 |
| Active | 0.1 | 0.1 | 0.1 | 0.1 | 0.1 |
| ingredient | in the | in the form | in the form | in the form | in the form |
| | unsalified | salified by the | salified by | salified by | salified by |
| | form | base added | the base | the base | the base |
| | | | added | added | added |
| Propylene | 75 | 75 | 75 | 75 | 75 |
| glycol | | | | | |
| Ethanol | 5 | 5 | 5 | 5 | 5 |
| Water | 20 | 18 | 18 | 18 | 18 |
| L-lysine | | 1.02 | | | |
| | | equivalents | | | |
| Lithium | | | 1.02 | | |
| hydroxide | | | equivalents | | |
| Sodium | | | | 1.02 | |
| hydroxide | | | | equivalents | |
| Triethanolamine | | | | | 1.02 |
| | | | | | equivalents |

The active ingredient concentrations are measured at times 0, 15 and 28 days and are given in the Table below:

| | | Concen- | Concen- | Concen- |
|---|---|---|---|---|
| | Theoretical | tration | tration | tration |
| | concentration | (in %) | (in %) at 15 | (in %) at 28 |
| Formulation | (% m/m) | at time 0 | days | days |
| Formulation 1 | 99 | 98 | 64 | 47 |
| Formulation 2 | 103 | 98 | 95 | 97 |
| Formulation 3 | 99 | 99 | 98 | 99 |
| Formulation 4 | 99 | 100 | 96 | 98 |
| Formulation 5 | 99 | 100 | 96 | 99 |

The results show an excellent chemical stability in the cases where the active ingredient is present in the salified form.

### 9.B. Physical and Chemical Stability of Example 2: Aqueous Gel

| | |
|---|---|
| Physical stability after 1 month at RT and 45° C. | NNC¹ |
| Physical stability after 2 months at RT and 45° C. | NNC |

¹NNC = no noticeable change

| | | |
|---|---|---|
| Chemical stability at T0 | | 99.4% |
| Chemical stability after 1 month | RT | 97.7% |
| | T 45° C. | 97.6% |
| Chemical stability after 3 months | RT | 99.4% |
| | T 45° C. | 97.6% |

### 9.C. Physical and Chemical Stability of Example 3: Cyclodextrin Gel

| | |
|---|---|
| Physical stability after 1 month at RT and 45° C. | NNC |
| Physical stability after 2 months at RT and 45° C. | NNC |

| | | |
|---|---|---|
| Chemical stability at T0 | | 100.1% |
| Chemical stability after 1 month | RT | 100.0% |
| | T 45° C. | 99.6% |
| Chemical stability after 3 months | RT | 102.2% |
| | T 45° C. | 101.7% |

### 9.D. Physical and Chemical Stability of Example 5: Oil-in-Water Emulsion

| | |
|---|---|
| Physical stability after 1 month at RT and 45° C. | NNC |
| Physical stability after 2 months at RT and 45° C. | NNC |

| | | |
|---|---|---|
| Chemical stability at T0 | | 100.7% |
| Chemical stability after 1 month | RT | 98.8% |
| | T 45° C. | 98.4% |
| Chemical stability after 2 months | RT | 100.7% |
| | T 45° C. | 99.5% |
| Chemical stability after 3 months | RT | 98.7% |
| | T 45° C. | 97.6% |

All the compositions according to the invention described above have an excellent physical and chemical stability of the solubilized active ingredient.

### EXAMPLE 8

### Results Pertaining to the Release/Penetration of the Active Ingredient

The two gels of Examples 6 and 7 were tested by comparison with a simple glycolic gel having the formulation below, rich in propenetrating glycol, in order to evaluate the level of release and penetration of the active ingredient within the preferred formulations according to the invention.

Protocol:

The release/penetration of the active ingredient in vitro within the compositions according to the invention can be evaluated on whole human skin.

The test formulation is applied for 16 hours on glass diffusion cells (3 ml; 1 cm²). Whole skin free of dermatomas was used. The skin was fixed to a diffusion cell, the dermis being in contact with a physiological saline solution supplemented with 0.25% (w/w) of an emulsifier (reception liquid). The system was maintained in static mode (no renewal of the reception liquid over time).

Abdominal and/or mammary skin flaps originating from cosmetic surgery operations were used. The formulation is applied to these three different skin specimens at a rate of 10 mg of formulation per cm². The applications were performed without occlusion. As the applications were performed in duplicate, the formulations were applied 6 times in total.

When the applications are complete, the surface excess is removed for each diffusion cell and the reception liquid and the skin are sampled. The epidermis (including the stratum corneum) is separated from the dermis. For each test formulation, a total balance of the active principle is calculated, taking into account the excess and the amounts found in the skin and in the reception liquid. The concentrations of active principle are determined by HPLC with APCI/MS/MS detection (quantification limit: 1 ng·m⁻¹).

Simple Glycolic Gel:

| COMMERCIAL NAME | INCI NAME | % |
|---|---|---|
| PROPYLENE GLYCOL | Propylene glycol | 75.00 |
| PURE RECTIFIED ETHANOL | Alcohol | 5.00 |
| PURIFIED WATER | Water | 18.822 |
| L-LYSINE (50% SOLUTION) | Lysine | 0.078 |
| 2-Hydroxy-4-[3-hydroxy-3- | Active ingredient | 0.1 |
| (5,6,7,8-tetrahydro-5,5,8,8- | | |
| tetramethyl-2-naphthyl)-1- | | |
| propynyl]benzoic acid | | |
| KLUCEL HF | Hydroxy propyl cellulose | 1 |

Release/Penetration Results:

| | Amount of active ingredient found in the |
|---|---|
| Formulation | reception medium |
| Simple glycolic gel | 0.35 ± 0.06 μg (3.6% of the applied dose) |
| Gel according to Example 7 | 0.69 ± 0.25 μg (8.0% of the applied dose) |
| Gel according to Example 6 | 1.05 ± 0.13 μg (11.4% of the applied dose) |

The results show that, in addition to chemical stabilization of the active ingredient, the optimized formulations according to the invention increase the bioavailability of the active ingredient in the skin (by a factor of two to three, respectively, compared with the reference gel).

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A topically applicable, physically/chemically stable cosmetic/pharmaceutical composition which comprises an aqueous phase and an active phase, said aqueous phase comprising water and said active phase comprising at least one solubilized retinoid, an amount of at least one base effective to salify said at least one retinoid and a hydrophilic cosolvent.

2. The cosmetic/pharmaceutical composition as defined by claim 1, formulated as an aqueous gel.

3. The cosmetic/pharmaceutical composition as defined by claim 1, formulated as an oil-in-water emulsion.

4. The cosmetic/pharmaceutical composition as defined by claim 1, comprising a retinoid having the formula (Ia):

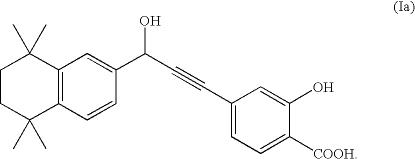

5. The cosmetic/pharmaceutical composition as defined by claim 1, said at least one base comprising a mineral and/or organic base and/or a basic amino acid.

6. The cosmetic/pharmaceutical composition as defined by claim 1, said at least one base comprising sodium hydroxide.

7. The cosmetic/pharmaceutical composition as defined by claim 1, comprising from 1 to 10 molar equivalents of said at least one base, based on said at least one retinoid.

8. The cosmetic/pharmaceutical composition as defined by claim 1, comprising from 0.01% to 5% by weight of said at least one retinoid.

9. The cosmetic/pharmaceutical composition as defined by claim 1, comprising from 40% to 80% by weight of water and from 5% to 20% by weight of a glycol cosolvent.

10. The cosmetic/pharmaceutical composition as defined by claim 1, comprising:
a) 0.1% of S-(+)-2-hydroxy-4-[3-hydroxy-3-(5,6,7,8-tetrahydro-5,5,8,8-tetramethylnaphthalen-2-yl)-1-propynyl]benzoic acid;
b) 2 molar equivalents of sodium hydroxide for salifying the retinoid;
c) 0.5% of Carbopol 980;
d) 65% to 75% of water; and
e) 15% of propylene glycol.

11. The cosmetic/pharmaceutical composition as defined by claim 1, comprising from 0.05% to 15% by weight of at least one gelling agent.

12. The cosmetic/pharmaceutical composition as defined by claim 1, comprising up to 15% by weight of at least one emulsifying agent.

13. A process for chemically stabilizing at least one solubilized retinoid within a cosmetic/pharmaceutical composition which comprises said at least one retinoid, a principal solvent and a cosolvent, comprising introducing therein an amount of at least one base effective to salify said at least one retinoid.

14. The process as defined by claim 13, said principal solvent comprising water.

15. The process as defined by claim 14, said at least one retinoid having the formula (Ia):

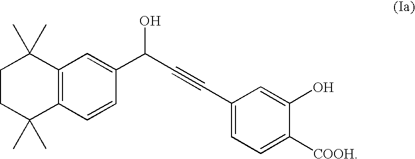

16. The process as defined by claim 14, said cosmetic/pharmaceutical composition comprising an aqueous gel.

17. The process as defined by claim 14, said cosmetic/pharmaceutical composition comprising an oil-in-water emulsion.

18. A regime or regimen for the prevention or treatment of a dermatological affliction or condition associated with cell differentiation and/or proliferation disorders and/or keratinization disorders, comprising topically applying onto the skin, integuments and/or mucosae of an individual in need of such treatment, a thus effective amount of the cosmetic/pharmaceutical composition as defined by claim 1.

19. A regime or regimen for the prevention or treatment of acne vulgaris or psoriasis, comprising topically applying onto the affected skin area of an individual in need of such treatment, a thus effective amount of the cosmetic/pharmaceutical composition as defined by claim 1.

20. A regime or regimen for treating skin prone to acne, for causing hair regrowth or preventing hair loss, for combating the greasy appearance of the skin or hair, in providing protection from the harmful effects of the sun, in treating physiologically dry skin or for preventing and/or combating photoinduced or chronological aging, comprising topically applying onto the affected skin/hair of an individual in need of such treatment, a thus effective amount of the cosmetic/pharmaceutical composition as defined by claim 1.
